(19) United States
(12) Patent Application Publication
(10) Pub. No.: US 2005/0031657 A1
(43) Pub. Date: Feb. 10, 2005

(54) Hydrosomes; chemical vessels for laser-guided microfluidics

(76) Inventors: Michael K. Gilson, North Potomac, MD (US); Kristian Helmerson, Darmestown, MD (US); Rani Kishore, Gaithersburg, MD (US)

Correspondence Address: INTELLECTUAL PROPERTY / TECHNOLOGY LAW, PO BOX 14329, RESEARCH TRIANGLE PARK, NC, 27709, US

(21) Appl. No.: 10/894,274

(22) Filed: Jul. 19, 2004

Related U.S. Application Data
(60) Provisional application No. 60/488,526, filed on Jul. 18, 2003.
(60) Provisional application No. 60/512,946, filed on Oct. 21, 2003.

(51) Int. Cl. A61K 7/00

(52) U.S. Cl. 424/401

(57) ABSTRACT

The present invention relates to a system for producing micro-vessels for holding small volumes of chemical reactants and fusing same to form microreactors and method of using same, wherein the micro-vessels are micron sized, surfactant stabilized water droplets called hydrosomes.

### CROSS-REFERENCE TO RELATED APPLICATIONS

This application claims priority from U.S. Provisional Patent Application Nos. 60/488,526 filed on Jul. 18, 2003 and 60/512,946 filed on Oct. 21, 2003 in the names of Michael K. Gilson, Kristian Helmerson, and Rani Kishore for “HYDRSOMES; CHEMICAL VESSELS FOR LASER-GUIDED MICROFLUIDICS” the contents of which are incorporated by reference herein for all purposes.

### BACKGROUND OF THE INVENTION

1. Field of the Invention

The present invention relates to microreactors, and more particularly, to systems for generating hydrosomes containing at least one chemical reactant and fusing the hydrosomes to create a microreactor and methods of using same for a controlled chemical reaction.

2. Description of the Related Art

The push to develop micro-fluidic technologies is driven by the needs of the analytical chemistry community for techniques that can rapidly process samples, which involve chemical reactions. Rapid processing requires both the expeditious handling of the samples and that the chemical reactions take place quickly. Both of these requirements can be satisfied by working with very small quantities of substances. The physical movement of small volumes of fluid and diffusional mixing of chemicals in these small volumes can be quite rapid. In addition, working with smaller quantities of chemicals makes combinatorial chemistry, for example for drug development, more cost effective.

In order to work effectively with ultra-small quantities, the substances must be held in correspondingly small containers or vials. In analogy to microtitre vials, nanovials have been fabricated in a various materials using a variety of techniques. The smallest nanovials hold hundreds of picoliters. These vials are, however, open containers. In order to hold smaller quantities, a closed container will be required in order to avoid rapid evaporation. Hence, a nanovial for handling pico-to femtoliters quantities should satisfy three main requirements. (1) The container should be closed or sufficiently isolated from the environment such that the substances held in the container do not escape into the surrounding medium, for example, by evaporation or diffusion. (2) The container must be able to be opened or, alternatively, controlled access to the interior of the container must be available. This is important so that reagents may be added or removed on demand in order to perform controlled chemical reactions within the containers. (3) The conditions in each container should be controlled independently to a sufficient degree such that individual reactions can take place in separate containers.

The three criteria for ultra-small volume containers for performing controlled chemical reactions are routinely satisfied by cells. Motivated by nature's solution, several groups are investigating the use of giant (cell sized) liposomes as nanofluid containers. Liposomes are closed structures consisting of a phospholipids bilayer membrane, which isolates the aqueous interior region from the external aqueous environment. The bilayer membrane can be either unilamellar or multilamellar. A number of experiments, demonstrating the use of liposomes as nanovials have been performed; however, there are a number of problems associated with the use of liposomes. The most notable problems are the lack of a technique for forming relatively uniform size liposomes, quickly and on demand, and the difficulty in incorporating reagents inside the liposomes in known quantities.

Thus, it would be advantageous to develop a microreactor that overcomes the shortcomings of the prior art.

### SUMMARY OF THE INVENTION

The present invention relates to a system for generating substantially uniform hydrosomes wherein the hydrosomes comprise reactive components and fusing of the hydrosomes forming an enclosed vessel for individual reactions therein. A preferred embodiment of the instant invention is a hydrosome comprising micron or less sized, surfactant stabilized water droplets in a fluorocarbon environment.

In one aspect, the present invention relates to a system for creating a microreactor, the system comprising:
a) a first container for holding an aqueous solution comprising at least one chemical component solubilized therein;
b) a second container for holding an organic solvent;
c) a droplet generator in fluid communication with the first and second container and positioned therebetween to generate droplets of the aqueous solution for ejection of same into the second container holding the organic solvent thereby generating hydrosomes; and
d) at least one source of electromagnetic energy positioned to direct electromagnetic energy at the second container to manipulate the hydrosomes for fusing together to form the microreactor, wherein the hydrosomes have a different refractive index from that of the organic solvent.

In another aspect, the present invention relates to a system for creating and fusing hydrosomes, the system comprising:
a) a first container for holding an aqueous solution comprising at least one chemical component solubilized therein and a surfactant;
b) a second container for holding an organic solvent;
c) a droplet generator in fluid communication with the first and second container and positioned therebetween, wherein the droplet generator device comprises:
   i) a plurality of nozzles in fluid communication with the first and second container wherein the nozzles comprise an aperture bore diameter sized to generate droplets of the aqueous solution; and
   ii) a pressure producing means communicatively contacting the aqueous solution to cause an increased pressure within the aqueous solution thereby generating droplets of the aqueous solution and causing the ejection of same through the nozzles into the second container holding the organic solvent thereby generating hydrosomes; and
d) at least one source of electromagnetic energy positioned to direct electromagnetic energy at the second container to manipulate the hydrosomes for fusing together therein.

In this embodiment, the pressure producing means may include a heating means to heat a portion of the aqueous solution to increase the pressure therein sufficiently to create expansion of the aqueous solution through a nozzle. Preferably, the pressure is sufficient to create a bubble in the aqueous solution thereby causing expansion of the aqueous solution through the nozzle. In the alternative, the pressure producing means may include a piezoelectric transducer that upon application of a voltage thereto, the transducer creates a vibration within the aqueous solution to displace the aqueous solution through the nozzles thereby creating a droplet ejection into the second container.

In yet another aspect, the present invention relates to a method for mixing reactive chemical components together, the method comprising:
a) solubilizing at least one chemical component in an aqueous solution comprising a surfactant;
b) generating droplets of the aqueous solution by applying sufficient pressure through mechanical and/or thermal means, to the aqueous solution to increase pressure therewithin in a sufficient amount to eject the aqueous solution through a plurality of nozzles thereby generating droplets of the aqueous solution;
c) introducing the droplets of the aqueous solution into an organic solvent thereby forming hydrosomes; and
d) manipulating the hydrosomes with electromagnetic energy to bring the hydrosomes together for fusing together.

In still a further aspect, the present invention relates to a method of mixing at least two chemical reactants comprising the steps of:
a) forming hydrosomes containing separately at least two chemical reactants, wherein the hydrosomes are formed by the steps comprising:
   i) solubilizing the chemical reactants in an aqueous solution, optionally containing a surfactant;
   ii) generating droplets of the aqueous solution and introducing the droplets of the aqueous solution into an organic solvent thereby forming hydrosomes wherein the organic solution has a different refractive index from that of the hydrosomes; and
b) bringing the at least two hydrosomes together such that they spontaneously fuse and mix the at least two chemical reactants, wherein the hydrosomes are brought together in either a simultaneous or sequential mode to induce ordered spontaneous fusing.

The droplets may be generated by any method that forms pico to micron-sized droplets with consistent and reproducible volumes including;
i) introducing the aqueous solution into an organic solvent, wherein the organic solvent comprises a high density than the aqueous solution and that is not miscible with the aqueous solution, and wherein upon mixing the aqueous solution separates from the organic solvent and disperses as individual droplets on or near the surface of the organic solution thereby forming hydrosomes; and
ii) applying sufficient pressure through mechanical and/or thermal means, to the aqueous solution to increase pressure therewithin in a sufficient amount to eject the aqueous solution through a plurality of nozzles or holes thereby generating droplets of the aqueous solution for introduction into the organic solvent.

Other aspects and features of the invention will be more fully apparent from the ensuing disclosure and appended claims.

### BRIEF DESCRIPTION OF THE FIGURES

FIG. 1 is a block diagram setting forth the basic components of one embodiment of the system of present invention illustrating the first and second containers, droplet generator electromagnetic energy source.

FIGS. 2A-2C are cross-sectional views of another embodiment of a droplet generator and ejector device of the present invention illustrating use of a heating means or piezoelectric material to provide sufficient pressure to generate and eject a droplet of the aqueous solution into the second container.

FIGS. 3A and 3B show hydrosomes containing Rhodamine B or fluorescently-labeled bovine serum albumin.

FIG. 4 shows fusion of two hydrosomes held in optical tweezers.

FIGS. 5A, 5B and 5C, FIG. 5A shows an image of the 3 μm diameter hydrosome and the ˜1 μm diameter dye containing hydrosome flowing towards it. (The hydrosome containing unbleached dye is indicated by the arrow.) FIGS. 5B and 5C are images, of the fluorescence from the hydrosomes when illuminated by 488 nm light. In FIG. 5B, the image is taken after the dye has photobleached, in FIG. 5C, the image is taken 2 minutes after the 3 μm diameter hydrosome has fused with a ˜1 μm diameter, dye containing hydrosome.

### DETAILED DESCRIPTION OF THE INVENTION

The term “solubilized” as used herein means a chemical compound is miscible in an aqueous solution and to form a substantially homogeneous solution.

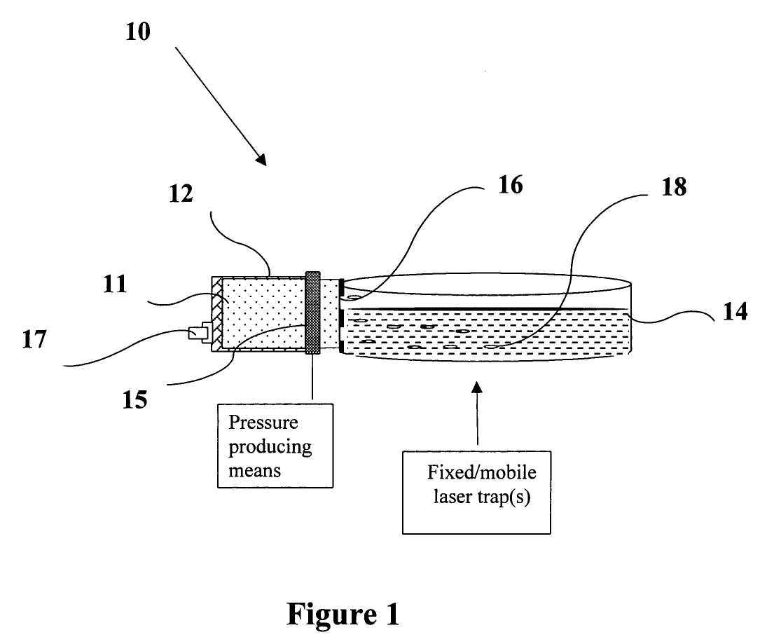

A hydrosome creating system in accordance with one embodiment of the present invention and illustrated in FIG. 1 overcomes the deficiencies of prior art systems and generates a controlled flow rate and droplet size of hydrosomes thereby providing uniform vessels for chemical reactants and upon fusing uniform microreactors for controlled chemical reactions. The system 10 comprises an aqueous solution container (first container) 12, for holding the aqueous solution 11. The first container is generally fabricated of a suitable material that will not react with the chemical components solubilized in the aqueous solution.

Positioned adjacent to the first container 12 and in fluid communication therewith is a second container 14 for holding an organic solution that has a greater density than the aqueous solution. Positioned between the first container 12 and second container 14 is a droplet generator 15 for producing water droplets for ejection into the second container.

In the basic configuration, the droplet generator 15 comprises at least one nozzle 16, and more preferably, a plurality of holes or nozzles in fluid communication with an aqueous solution retained in the first container. The nozzle configuration is shaped to provide effective and unencumbered flow of the aqueous solution therethrough, and can include applicable configurations such as circular, elliptical, rectangular and the like. The nozzle geometry directly effects droplet volume and ejection velocity, and as such, the aperture bore diameter and geometry should be considered when determining requirements of droplet sizes and frequency of formation. Because small droplet volume is preferred to achieve smaller volume vessels, the nozzle aperture bore diameter is preferably from about 0.0001 um to about 100 um, and more preferably from about 0.010 to 10 um.

The quantity of nozzles incorporated into a droplet generator of the present invention is determined by the volume of each droplet, the velocity of the ejected drop, the refill flow rate into the nozzle area, the viscosity of the aqueous solution, whether a surfactant is included in the aqueous solution, and the required flow rate of droplets into the second container. Preferably, the number of nozzles ranges from about 20 to about 400 per device. which, depending on the viscosity of the aqueous solution and droplet size, can generate from about 500 drops per second up to about 6000 drops per second.

The droplet generator of the present invention further comprises a pressure producing means 15 to effectuate ejection of the aqueous solution through the plurality of nozzles. In the FIG. 1, the pressure producing means may comprises a heating element that can rapidly heat the aqueous solution in a sufficient amount to cause an increase in pressure within the contained solution.

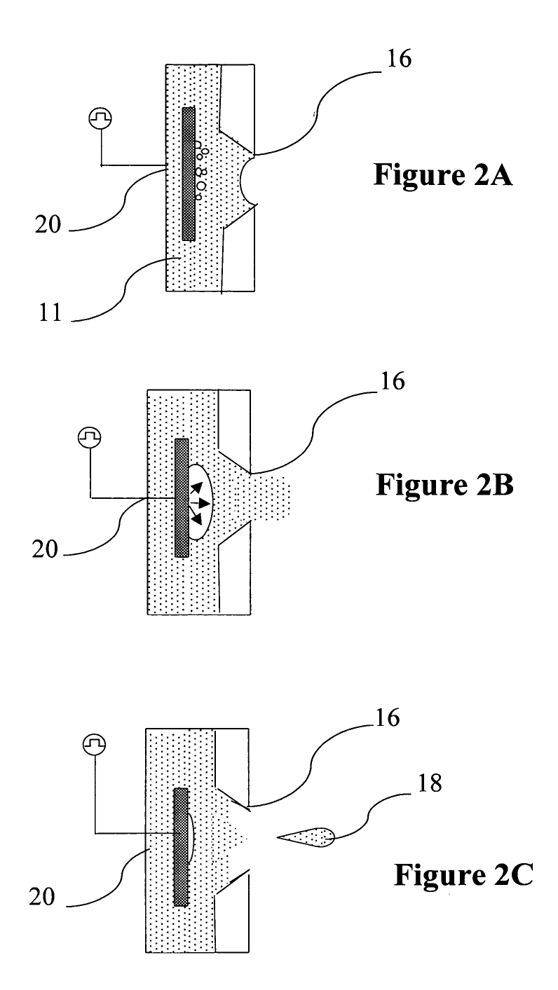

The heating element may include resistive heating systems, block heaters or induction heating devices. The heating element may be selectively activated through an electrode setup 17, which is in electrical contact with the heating element. In use, a continuous current or a current pulse (periodic) of less than a few microseconds through the heater causes heat to be transferred from the surface of the heater to the aqueous solution. The aqueous solution is preferably heated to the critical temperature for bubble nucleation as shown in FIG. 2A. When nucleation occurs, vapor bubbles instantaneously expand to force the aqueous solution into the nozzle, as shown in FIG. 2B. The increased pressure within the aqueous solution and first container has to be greater than that within the second container. s the bubble collapses, the increased pressure within the first container is reduced and a droplet 18 of aqueous solution breaks off and enters into the second container, as shown in FIG. 2C. This entire process can occur in the range from about 10 us to about 50 us depending on the heater temperature, viscosity of the aqueous material and volume of the droplet. Further, the volume of each droplet, which is again dependent on the aperture bore size and configuration, can be in the range from about 0.001 to about 100,000 picoliters.

In the alternative, the droplet generator 15 (See FIG. 1) comprises an electromechanical transducer, as the pressure producing means, which generates a vibrational pressure wave to increase pressure within the aqueous solution. The most popular type of electromechanical transducers uses the piezoelectric effect. The piezoelectric effect occurs in several natural and artificial crystals and is defined as a change in the dimensions of the crystal when an electric charge is applied to the crystal faces. The importance of the piezoelectric effect is that the piezoelectric material provides a means of converting electrical oscillations into mechanical oscillations.

Any commonly used piezoelectric material may be utilized in the present invention including, but not limited to, modified lead titanate, quartz, barium titanate, lithium sulfate, lead-zirconate-titanate, and lead niobate. Examples of acoustic transducers which are commercially available and may be used in this present invention include: Matec broadband MIBO series (5-10 MHZ), Matec broadband MICO (3.5 MHZ), Matec broadband MIDO 2.25 MHZ), and Matec broadband MIEC series (50 kHz-1 MHZ).

The geometry of transducer utilized in this invention can be any shape, such as circular or rectangular (linear arrays). It is important to note that in using a piezoelectric transducer the output from a separate variable-frequency oscillator or signal generator does not have to be applied to the transducer. The transducer can actually be part of the oscillator circuit itself, and it is the chosen resonance frequency of the piezoelectric crystal that stabilizes the frequency of the electrical oscillations. Applicable transducers will include types that produce vibrational acoustic wave within a range of frequencies (broadband) or for one specific frequency (narrowband) for frequencies ranging from hertz to gigahertz. Any solid-state pulser or microprocessor can control the pulse duration in the present invention.

In use, the piezoelectric transducer undergoes deformation when an electrical signal generates a mechanical strain within the piezoelectric material. The piezoelectric material expands or bends and applies pressure against the diaphragm, which in turn applies pressure to the source material. The deformation of the piezoelectric material causes an increased pressure within the aqueous solution generating a pressure wave that propagates toward the nozzle to form a droplet of the aqueous solution ejected at the nozzle 16. Because the deformation of a piezoelectric transducer is on the submicron scale, the size of the piezoelectric transducer should preferably be of sufficient size to cause enough volume displacement to form a droplet. As such, the piezoelectric transducer preferably is at least as large as the bore diameter of the nozzle, and more preferably, at least twice the size of the bore diameter.

The droplet generators of the present invention have the advantages of introducing a controlled and reproducible amount of aqueous solution into the organic solution in the second container thereby ensuring consistency in the droplet size and volume of chemical components in the formed hydrosome. The formed hydrosomes may contain different chemical components and as such, multiple droplet generators may be used to introduce a plurality of hydrosomes containing chemical reactants that when combined or fused provide for a microreactor for a subsequent chemical reaction.

In another embodiment, the aqueous solution may be introduced directly into the organic solution and droplets generated by sonification of the mixture. Hydrosomes are formed thereby encapsulating the water droplet and solubilized chemical components therein. However, this method does not produce droplet of reproducible volumes and the drop volumes can vary widely.

The hydrosomes of the present invention are formed when introducing an aqueous solution comprising a chemical component into an organic solution, wherein the organic solution or solvent is not miscible with the aqueous solution. The chemical component may include any compound that can be solubilized in an aqueous solution including, but not limited to a pharmaceutically active compounds, electrolytes, substances that activates receptors on the cell plasma membrane, agents that affects intracellular chemistry, agents that affects cellular physics, genes, gene analogs, RNA, RNA analogs, DNA, DNA analogs, colloidal particles, receptors, receptor ligands, receptor antagonists, receptor blockers, enzymes, enzyme substrates, enzyme inhibitors, enzyme modulators, proteins, protein analogs, amino acids, amino acid analogs, peptides, peptide analogs, metabolites, metabolite analogs, oligonucleotides, oligonucleotide analogs, antigens, antigen analogs, haptens, hapten analogs, antibodies, antibody analogs, organelles, organelle analogs, cell nuclei, bacteria, viruses, gametes, inorganic ions, metal ions, metal clusters, polymers, fluorescent compounds and any combinations thereof.

Preferably the aqueous solution further comprises a surfactant, which has the ability to stabilize the water droplets and reduce droplet evaporation. Any surfactant that stablizes the generated droplets and provides a stable interface between the aqueous droplets and organic solvent may be used in the present invention. As used herein, the term “surfactant” refers to a surface-active agent that generally comprises a hydrophobic portion and a hydrophilic portion. Surfactants may be categorized as anionic, nonionic, amphoteric, or cationic, depending on whether they comprise one or more charged groups.

Anionic surfactants, such as SDS or lauryl sarkosine, contain a negatively charged group and have a net negative charge. Nonionic surfactants contain non-charged polar groups and have no charge. Exemplary nonionic surfactants include, but are not limited to, t-octylphenoxypolyethoxyethanol (Triton X-100), polyoxyethylenesorbitan monolaurate (Tween 20), polyoxyethylenesorbitan monolaurate (Tween 21), polyoxyethylenesorbitan monopalmitate (Tween 40), polyoxyethylenesorbitan monostearate (Tween 60), polyoxyethylenesorbitan monooleate (Tween 80), polyoxyethylenesorbitan monotrioleate (Tween 85), (octylphenoxy)polyethoxyethanol (IGEPAL CA-630/NP-40), triethyleneglycol monolauryl ether (Brij 30), and sorbitan monolaurate (Span 20). An amphoteric surfactant contains both a positively charged group and a negatively charged group, and has no net charge.

A “cationic surfactant” has a positively charged group under the conditions examined. Cationic surfactants may contain quaternary amines or tertiary amines. Exemplary quaternary amine surfactants include, but are not limited to, cetylpyridinium chloride, cetyltrimethylammonium bromide (CTAB; Calbiochem #B22633 or Aldrich #85582-0), cetyltrimethylammonium chloride (CTACl; Aldrich #29273-7), dodecyltrimethylammonium bromide (DTAB, Sigma #D-8638), dodecyltrimethylammonium chloride (DTACl), octyl trimethyl ammonium bromide, tetradecyltrimethylammonium bromide (TTAB), tetradecyltrimethylammonium chloride (TTACl), dodecylethyidimethylammonium bromide (DEDTAB), decyltrimethylammonium bromide (DIOTAB), dodecyltriphenylphosphonium bromide (DTPB), octadecylyl trimethyl ammonium bromide, stearoalkonium chloride, olealkonium chloride, cetrimonium chloride, alkyl trimethyl ammonium methosulfate, palmitamidopropyl trimethyl chloride, quaternium 84 (Mackernium NLE; McIntyre Group, Ltd.), and wheat lipid epoxide (Mackernium WLE; McIntyre Group, Ltd.). Exemplary ternary amine surfactants include, but are not limited to, octyldimethylamine, decyidimethylamine, dodecyidimethylamine, tetradecyldimethylamine, hexadecyidimethylamine, octyldecyldimethylamine, octyidecylmethylamine, didecylmethylamine, dodecylmethylamine, triacetylammonium chloride, cetrimonium chloride, and alkyl dimethyl benzyl ammonium chloride.

In a preferred embodiment, at least one nonionic surfactant is used and selected from selected from the group comprising t-octylphenoxypolyethoxyethanol (Triton X-100), polyoxyethylenesorbitan monolaurate (Tween 21), polyoxyethylenesorbitan monopalmitate (Tween 40), polyoxyethylenesorbitan monostearate (Tween 60), polyoxyethylenesorbitan monooleate (Tween 80), polyoxyethylenesorbitan monotrioleate (Tween 85), (octylphenoxy)polyethoxyethanol (IGEPAL CA-630), triethyleneglycol monolauryl ether (Brij 30), and sorbitan monolaurate (Span 20).

The chemical components and preferably the surfactant are added to water and solubilized therein to prepare the aqueous solution for subsequent droplet formation. As discussed above, droplet formation may include several methods including a droplet generating device using a pressure inducing mechanism using either mechanical or thermal energy.

Additional components may be included in the aqueous solution including buffers that may be defined as compositions that resist changes in pH when acids or bases are added to the solution. This resistance to pH change is due to the solution's buffering action. Solutions exhibiting buffering activity are referred to as buffers or buffer solutions. Buffers typically do not have an unlimited ability to maintain the pH of a solution or composition. Rather, typically they are able to maintain the pH within certain ranges, for example between pH 5 and pH 7. See, generally, C. Mohan, Buffers, A guide for the preparation and use of buffers in biological systems, Calbiochem, 1999. Exemplary buffers include, but are not limited to, MES ([2-(N-Morphilino)ethanesulfonic acid]), ADA (N-2-Acetamido-2-iminodiacetic acid), and Tris ([tris(Hydroxymethyl)aminomethane]; also known as Trizma); Bis-Tris; ACES; PIPES; MOPS; and the like (all available from Sigma).

The generated droplets are ejected into a second container 14 that comprises an organic solvent. As used herein, the term “organic solvent” refers to organic liquids, i.e., those comprising molecules with a hydrocarbon backbone and having a higher density than the aqueous solution. Any organic solvent may be used that upon mixing with the aqueous solution provides for separation of aqueous droplets from the organic solution and allows individual droplets to form as hydrosomes on or near the surface of the organic solvent, thereby forming a layer of hydrosomes.

The organic solvent, may be any suitable solvent that is liquid at room temperature, preferably a fluorinated hydrocarbon solvent. More preferably, the solvent is a highly fluorinated solvent, especially a branched or unbranched, cyclic or non-cyclic lo fluoroalkane. Most preferably, the solvent is perfluorinated. The term “perfluorinated solvent” as used herein includes organic compounds in which all (or essentially all) of the hydrogen atoms are replaced with fluorine atoms. The perfluorinated solvents can be perfluoroaliphatic compounds, having 5 to 18 carbon atoms, optionally containing one or more catenary heteroatoms, such as divalent oxygen or trivalent nitrogen and include perfluoroalkanes (occasionally referred to as PFCs). Useful perfluorinated solvents include the following: perfluoropolyether, perfluoropentane, perfluorohexane, perfluoroheptane, perfluorooctane, perfluorotributyl amine, perfluorotriamyl amine, perfluoro-N-methylmorpholine, perfluoro-N-ethylmorpholine, perfluoro-N-methyl pyrrolidine, perfluoro-1,2-bis(trifluoromethyl)hexafluorocyclobutane, perfluoro-2-butyltetrahydrofuran, perfluorotriethylamine, perfluorodibutyl ether, and mixtures of these and other perfluorinated liquids. Commercially available perfluorinated solvents that can be used as these solvents include: Fluorinert™ FC-43, Fluorinert™ FC-70, Fluorinert™ FC-72, Fluorinert™ FC-77, Fluorinert™ FC-84 and Fluorinert™ FC-87 (Fluorinert™ Liquids, product bulletin 98-0211-6086(212)NPI, issued 2/91, available from 3M Co., St. Paul, Minn.)

When the droplets of aqueous solution are introduced into the organic solvent, the aqueous droplets encapsulate the chemical compounds forming substantially uniform micro-vessels with substantially reproducible volumes of chemical components. The hydrosomes are then manipulated in the fluorocarbon background to bring at least two hydrosomes into contact with each other, which allows them to fuse and form a single hydrosome. The fusing process results in a mixing of the contents of the individual hydrosomes in a manner that allows for a controlled chemical reaction involving small quantities of reagents.

The means for manipulating the hydrosomes on or in the organic solvent may involve the application of optical, thermal, acoustic, electric field, magnetic, and/or physical forces. Preferably, the manipulation of the individual hydrosomes is accomplished by using optical forces and the gradient force that exists when a transparent material with a refractive index greater than the surrounding medium is placed in a light gradient. As light passes through polarizable material, it induces a dipole moment. This dipole interacts with the electromagnetic field gradient, resulting in a force directed towards the brighter region of the light, normally the focal region. Optical traps, sometimes referred to as “optical tweezers,” utilize a light source to produce radiation pressure. Radiation pressure is a property of light that creates small forces by absorption, reflection, or refraction of light by a dielectric material. Relative to other types of forces, the forces generated by radiation pressure are almost negligible—only a few picoNewtons (1 pN=1×10-12 N) from a light source of a few milliwatts of power. However, a force of a few picoNewtons is more than sufficient to represent the interactions of microscopic molecules. Optical traps utilize brighter regions or focal points of light to draw the hydrosome toward the direction of the focal region of the light source because the hydrosome minimizes its energy by moving to the region where the electric field is the highest, namely the focal point of the laser beam.

A simplified model of the optical trap is as follows: light, such as laser light, enters a high numerical aperture objective lens of an optical system and is focused to a diffraction-limited region or spot on a spherical object in the specimen plane. Because the intensity profile of the laser light is not uniform, an imbalance in the reaction forces generates a three-dimensional gradient force with the brightest light in the center. The gradient force pulls the object toward the brighter side. Thus, the picoNewton forces generated by the optical system “traps” the object. Such gradient forces are formed near any light focal region.

The sharper or smaller the focal region, the steeper the gradient. To overcome scattering forces near the focal region and hence prevent the object from being ejected along the direction of the light beam, the optical system must produce the steepest possible gradient forces. Sufficiently steep gradient forces can be achieved by focusing laser light to a diffraction-limited spot of diameter of approximately λ, the laser light wavelength, through a microscope objective of high numerical aperture (N.A).).

Generally, any source of electromagnetic radiation may be employed with the invention as long as the source generates electromagnetic radiation capable of optically moving the hydrosomes. In general, more intense light provides larger forces, and hence larger accelerations and faster motion. The use of a highly focused laser beam, first realized by Ashkin in 1986, usually referred to as the Optical Tweezer, has been studied extensively (Ashkin A., Dziedizic J. M., Bjorkholm J. E., Chu S., “Observation of a Single-Beam Force Optical Trap for Dielectric Particles” Opt. Lett. 11(5), p.288-290 (1986); Ashkin A., “Forces of a Single-Beam Gradient Laser trap on a Dielectric Sphere in the Ray Optics Regime” Biophys. J. 61, p.569-582 (1992); Wright W. H., Sonek G. J., Bems M. W., “Radiation Trapping Forces on Microspheres with Optical Tweezers” App. Phys. Lett. 63, p.715-717 (1993); Gussgard R., Lindmo T., Brevik I., “Calculation of the Trapping Force in a Strongly Focused Laser Beam” J. Opt. Soc. Am. B 9(10), p.1922-1930 (1992); Visscher K., Brakenhoff G. J., “Theoretical Study of Optically Induced Forces on Spherical Particles in a Single Beam Trap I: Rayleigh Scatterers” Optik 89(4), p.174-180 (1992); Kuo S. C., Sheetz M. P., “Optical Tweezers in Cell Biology” Trends in Cell Biology 2, p.116-118 (1992)) and is widely used in biological applications.

For detailed review of applicable setups for instrumentation, see Kuo S. C., Sheetz M. P., “Optical Tweezers in Cell Biology” Trends in Cell Biology 2, p.116-118 (1992); Bems M. W., Wright W. H., Steubing R. W., “Laser Microbeam as a Tool in Cell Biology” Int. Rev. Cytol. 129, p.1-44 (1991); Block S. M., Optical Tweezers: A new Tool for Biophysics, in Noninvasive Techniques in Cell Biology, G. S. Foskett J. K., Editor. 1990, Wiley-Liss.: New York. p. 375-402; Greulich K. O., Weber G., “The Laser Microscope on its Way from an Analytical to a Preparative Tool” J. Microsc. 167, p.127-151 (1991); Simmens R. M., Finer J. T., “Glasperlenspiel II: Optical Tweezers” Curr. Biol. 3, p.309-311 (1993); Weber G., Greulich K. O., “Manipulation of Cells, Organelles, and Genome by Laser Microbeams and Optical Traps” Int. Rev. Cytol. 133, p.1-41 (1992); and Mammen, M., Helmerson, K. Choi, S., Phillips, W.D., Whitesides, G. M., Chem. Biol. (1996), 3, p. 757-763, the contents of which are hereby incorporated herein by reference for all purposes.

One area of concern is that the surface of a fluid droplet might deform while exposed to the trapping beam. However, at the low power levels applied continuously to the droplet, typically in the range of 1-200 mW, changes to the droplet are minimal. Typically, the particular wavelength corresponds to a wavelength that is minimally absorbed by the selected fluid. Light absorption tends to heat the droplets, enhances evaporative loss therefrom, and in some cases, may cause the droplets to degrade, particularly when the light is intense. According to Hale et al. (1973), “Optical constants of water in the 200-nm to 200-um wavelength region,” Appl. Opt. 12:555-563, water has a minimum absorption wavelength of about 480 nm. Accordingly, optical movement of aqueous fluids is generally preferred at or near this wavelength, with an adjustment relative to the light absorption wavelength of the chemical components therein. Preferably, the wavelength ranges from about 200 nm to about 700 nm. Light at this wavelength can be produced directly using laser diodes, or through frequency doubling the wavelength of an infrared laser diode.

Diode lasers are preferred because of their low power consumption and compact size. However, other continuous wave sources may be used, such as an argon laser, a Ti:sapphire laser, or a dye laser. Alternatively, a light emitting diode (LED) or superluminescent LED may be used.

As discussed above, a plurality of hydrosomes may be present on or near the surface of the organic solvent layer in the second container. Thus, the means for directing electromagnetic radiation may be adapted to optically move a plurality of droplets on the fluid-transporting surface. In some instances, the means for directing electromagnetic radiation may be adapted to optically move the droplets in succession. Alternatively, the means for directing electromagnetic radiation is adapted to optically move the droplets simultaneously.

Droplet size of the hydrosome is an important consideration for optical microfluidics. The droplet size influences the applied force, the relative sizes of the surface tension and volume forces, and the evaporation rate. Thus, the invention is suitable for any droplet having a size that allows for its movement through optical trapping forces. That is, depending on the interaction between the directed light and the fluid droplet, varying degrees of momentum transfer may take place. Through ordinary electromagnetic radiation sources, one can usually optically move droplets having a volume of about 10 nanoliters or less with a fair degree of repeatability and precision. Repeatability and precision is improved when the droplet has a reduced volume as in the present invention.

### EXAMPLE 1

Hydrosomes

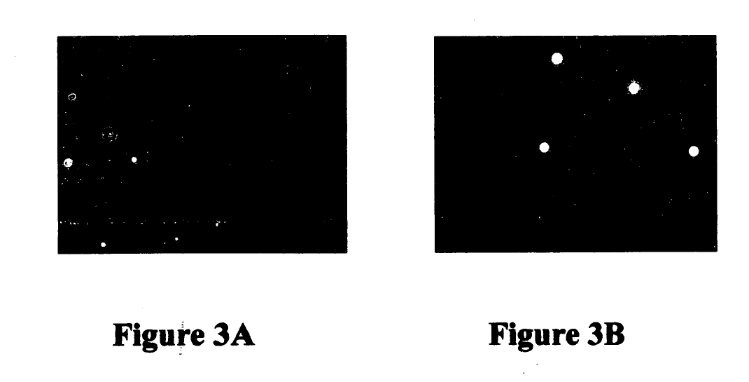

A number of problems associated with the reliable formation of liposomes and the incorporation of various chemicals in the liposomes can be circumvented using a novel system, called hydrosomes. Basically, a hydrosome is a water droplet (micron-sized) in a different background, chemical environment. In particular, in this embodiment the hydrosomes are water (or any aqueous buffer solution) droplets in a fluorocarbon, such as FLUORINERT (3M, Minneapolis, Minn.). The solubility of water in the fluorocarbon is at the level of a few ppm, thus the water droplets are essentially stable. Further stability, especially if the water droplets are at an elevated temperature or subjected to some heating mechanism, was achieved by using a surfactant, such as TWEEN-20 or TRITON, in the water. Formation of the water droplets was easily accomplished via ultrasonic agitation, which yields droplets with diameters in the range of 0.1 to 1 micron, or with microfluidic nozzles, which resulted in bigger droplets, but more mono-disperse in size. Substances that were solubilized in the water during formation of the hydrosomes are readily incorporated into the hydrosomes at the known concentration of the solution. FIGS. 3A and 3B is fluorescence microscopy images of the hydrosomes with either Rhodamine B dye or fluorescently labeled bovine serum albumin incorporated in them.

### EXAMPLE 2

Optical Manipulation of Hydrosomes

A single focus laser trap, or optical tweezers, was used to trap and remotely manipulate the hydrosomes. Optical tweezers rely on the increased polarizability of the object to be trapped compared to the surrounding medium, such that the energy of interaction between the object and the laser field is a minimum. That is, the object to be trapped must have an index of refraction higher than the surrounding medium. In the case of the present hydrosomes, the index of refraction of water compared to FLUORINERT, FC-77, is 1.33 to 1.29 therefore the hydrosomes are easily manipulated with optical tweezers. The apparatus consists of dual optical tweezers, both of which use light from infrared lasers (Nd:YAG, X=1.064 μm). One trap is fixed and the other can be swept across the field of view, so that two hydrosomes can be trapped simultaneously and moved with respect to each other. The trapping apparatus is based on a Zeiss inverted microscope. The microscope included laser-induced fluorescence using either a fiber-coupled argon ion laser or diode lasers, such that a broad range of fluorescent dyes can be used; and low light level detection using an image intensified CCD camera so that chemical reactions involving only a few number of molecules can be studied.

### EXAMPLE 3

Fusion of Hydrosomes

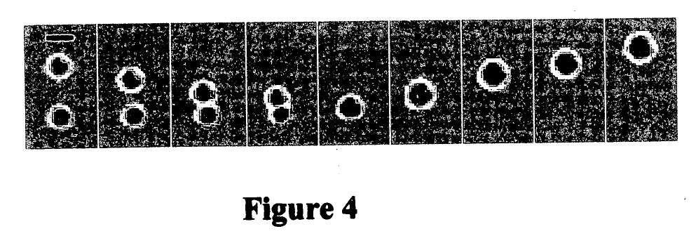

Fusion of hydrosomes differs from that of liposomes in two major ways. First, unlike liposomes where fusion has to be induced, hydrosomes tend to fuse spontaneously when they are brought into contact. The significance of this is that it greatly simplifies any device that might use hydrosomes in an assay (no external device such as microelectrodes or a pulsed laser beam is needed). Second, fusion of hydrosomes does not involve any rearrangement of a membrane, which is necessary for liposomes. Therefore the whole fusion process may happen faster and mixing of reagents may occur faster, which would greatly speed up chemical reactions and sample processing. FIG. 4 shows a sequence of video images showing the fusion of two hydrosomes that were held in independent optical tweezers.

### EXAMPLE 4

Demonstration of a Controlled Reaction

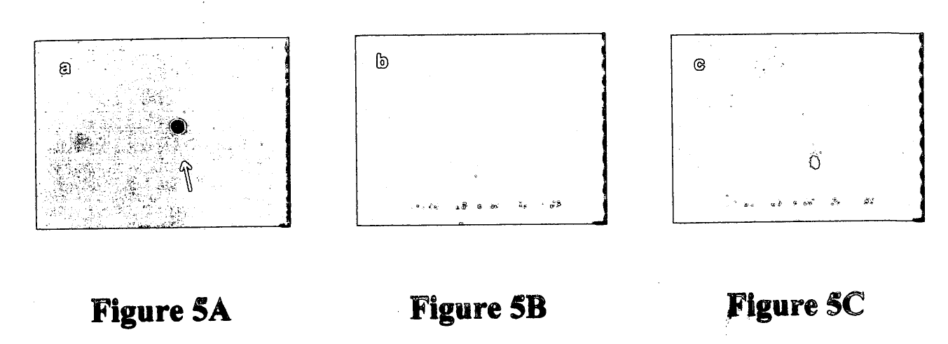

In order to demonstrate the applicability of hydrosomes as ultra-small volume containers for chemistry, a controlled reaction was performed by fusing two hydrosomes, allowing their individual contents to mix. The reaction demonstrated the binding of an intercalating dye to DNA. A 3 μm diameter hydrosome was prepared by randomly fusing a collection of ˜1 micron diameter hydrosomes. The smaller hydrosomes contained either DNA ladder segments ˜1 kbp in length or YOYO-1 intercalating dye. The concentration of the DNA ladder segments in solution before forming the hydrosomes was 10 mg/L, which implies that a 1 μm diameter hydrosome contains approximately 5 DNA ladder molecules or a total of about 5,000 base pairs. The concentration of YOYO-1 in solution prior to forming the hydrosomes was 4 μm, which implies that a 1 μm diameter hydrosome contains approximately 1000 dye molecules. We verified that the individual hydrosomes, either the ones containing only DNA or only dye, did not fluoresce when excited by 488 nm light. The 3 μm hydrosome did fluoresce and hence it contained both DNA and dye, which was expected since it was prepared from a random selection of hydrosomes. it Based on the random selection it was expected that the 3 μm hydrosome contained 70±35 DNA molecules. The controlled reaction consisted of the following procedure. The 3 μm diameter hydrosome containing both DNA and dye was held in an optical trap. It was then exposed to the 488 nm light. The fluorescence was monitored and observed to extinguish below a detectable level in approximately 15 seconds due to photobleaching. Subsequent excitation, even if the hydrosome was first kept in the dark for up to 30 minutes, produced no detectable fluorescence. Next, ˜1 μm diameter hydrosomes, containing only YOYO-dye, were transported under flow at a velocity of 10 μm /s through the region containing the 3 μm hydrosome. The trapped, 3 μm hydrosome was maneuvered in order to intersect the path of a dye containing hydrosome. When the two hydrosomes nearly overlapped, the optical trap pulled the dye containing hydrosome into contact with the 3 μm hydrosome and the two hydrosomes subsequently fused. The composite hydrosome was held in the optical trap for 2 minutes before re-exposure to the 488 nm light. Fluorescence was observed to emanate from the composite liposome. A minimum of 30 seconds was required after fusion before any fluorescence could be detected. This is presumably because the dye needs time to intercalate in the DNA segments. (The mixing time is negligible on this timescale.) After the fluorescence had extinguished due to continued excitation, the controlled reaction was repeated by fusing the composite hydrosome with another hydrosome containing only dye. FIG. 5A shows an image of the 3 μm diameter hydrosome and the ˜1 μm diameter dye containing hydrosome flowing towards it. (The dye containing hydrosome is indicated by the arrow.) FIGS. 5B and 5C are images of the fluorescence from the hydrosomes when illuminated by 488 nm light. In FIG. 5B, the image was taken after the dye had photobleached. In FIG. 5C, the image was taken 2 minutes after the 3 μm diameter hydrosome had fused with a ˜1 μm diameter, dye containing hydrosome.

The disclosure and examples are provided for illustration of the instant invention and are not intended to limit the scope of the invention. All references are herein incorporated in their entirety by reference.

1. A hydrosome comprising micron sized, surfactant stabilized water droplet(s) in a fluorocarbon environment.

2. The hydrosome of claim 1, wherein said water contains solubilized reactants.

3. The hydrosomes of claim 2, wherein said reactant comprises DNA, proteins, enzymes, or fluorescent compounds.

4. A method for mixing reactive chemical components together, the method comprising:
a) solubilizing at least one chemical component in an aqueous solution comprising a surfactant;
b) generating droplets of the aqueous solution by applying sufficient pressure through mechanical and/or thermal means, to the aqueous solution to increase pressure therewithin in a sufficient amount to eject the aqueous solution through a plurality of nozzles thereby generating droplets of the aqueous solution;
c) introducing the droplets of the aqueous solution into an organic solvent thereby forming hydrosomes; and
d) manipulating the hydrosomes with electromagnetic energy to bring the hydrosomes together for fusing together.

5. The method of claim 4, wherein the surfactant is selected from the group consisting of t-octylphenoxypolyethoxyethanol, polyoxyethylenesorbitan monolaurate, polyoxyethylenesorbitan monopalmitate, polyoxyethylenesorbitan monostearate, polyoxyethylenesorbitan monooleate, polyoxyethylenesorbitan monotrioleate, (octylphenoxy)polyethoxyethanol, triethyleneglycol monolauryl ether and sorbitan monolaurate.

6. The method of claim 4, wherein the organic solvent is a perfluorinated compound.

7. The method of claim 4, wherein the organic solvent has a higher density than the aqueous solution.

8. The method of claim 4, wherein the hydrosomes have a higher refractive index from that of the organic solvent.

9. The method of claim 4 wherein droplets of the aqueous solution are generated by applying mechanical pressure by using an electromechanical transducer which generates a vibrational pressure wave within the aqueous solution to form a droplet of the aqueous solution by ejection from a droplet forming nozzles.

10. The method of claim 9, wherein the electomechanical transducer is fabricated from a piezoelectric material.

11. The method according to claim 6, wherein the perfluorinated compound is perfluoropolyether.

12. The method of claim 4, wherein the chemical component comprises DNA, proteins, enzymes, or fluorescent compounds.

13. The method of claim 4, wherein the chemical component is selected from the group consisting of genes, gene analogs, RNA, RNA analogs, DNA, DNA analogs, colloidal particles, receptors, receptor ligands, receptor antagonists, receptor blockers, enzymes, enzyme substrates, enzyme inhibitors, enzyme modulators, proteins, protein analogs, amino acids, amino acid analogs, peptides, peptide analogs, metabolites, metabolite analogs, oligonucleotides, oligonucleotide analogs, antigens, antigen analogs, haptens, hapten analogs, antibodies, antibody analogs, organelles, organelle analogs, cell nuclei, bacteria, viruses, gametes, inorganic ions, metal ions, metal clusters, polymers, fluorescent compounds and any combinations thereof.

14. The method of claim 4, wherein manipulating the hydrosome with electromagnetic energy comprises the steps of providing a focused beam of laser light in a wavelength range to form conditions for an optical trap, the hydrosomes having a weak absorption coefficient in the wavelength range of the laser light such that the hydrosome does not absorb the energy wavelength which permits manipulation without substantial damage to the hydrosome.

15. The method of claim 14, wherein the focused beam of laser light includes light in the wavelength of about 300 nm to about 700 nm.

16. The method of claim 4, wherein the hydrosomes spontaneously fuse together when brought together and contact each other.

17. The method of claim 14, wherein the focused beam of laser light is continuous.

18. The method of claim 14, wherein the focused beam of laser light is pulsed.

19. The method of claim 14, wherein the focused beam of laser light is selected from the group consisting of the visible and ultraviolet wavelength ranges.

20. A method of mixing at least two chemical reactants comprising the steps of:
a) forming at least two hydrosomes containing separately different chemical reactants, wherein the hydrosomes are formed by the steps comprising:
i) solubilizing the chemical reactants in an aqueous solution, optionally containing a surfactant;
ii) generating droplets of the aqueous solution and introducing the droplets of the aqueous solution into an organic solvent thereby forming hydrosomes wherein the organic solution has a different refractive index from that of the hydrosomes; and
b) bringing the at least two hydrosomes together such that they spontaneously fuse and mix chemical reactants, wherein the hydrosomes are brought together in either a simultaneous or sequential mode to induce spontaneous fusing.

21. The method of claim 20, wherein the droplets are formed by introducing the aqueous solution into the organic solvent comprising a high density liquid that is not miscible with the aqueous solvent and wherein upon mixing the aqueous solution separates from the organic solvent and forms individual droplets thereby forming hydrosomes.

22. The method of claim 20, wherein the droplets are formed by applying sufficient pressure through mechanical and/or thermal means, to the aqueous solution to increase pressure therewithin in a sufficient amount to eject the aqueous solution through a plurality of nozzles or holes thereby generating droplets of the aqueous solution for introduction into the organic solution.

23. The method of claim 20, wherein bringing the at least two hydrosomes together such that they spontaneously fuse and mix chemical reactants comprises the steps of providing a focused beam of laser light in a wavelength range to form conditions for an optical trap, the hydrosomes having a weak absorption coefficient in the wavelength range of the laser light such that the hydrosomes do not absorb the energy wavelength which permits manipulation without substantial damage to the hydrosome.

24. The method of claim 20, wherein the surfactant is selected from the group consisting of t-octylphenoxypolyethoxyethanol, polyoxyethylenesorbitan monolaurate, polyoxyethylenesorbitan monopalmitate, polyoxyethylenesorbitan monostearate, polyoxyethylenesorbitan monooleate, polyoxyethylenesorbitan monotrioleate, (octylphenoxy)polyethoxyethanol, triethyleneglycol monolauryl ether and sorbitan monolaurate.

25. The method of claim 20, wherein the organic solvent is a perfluorinated compound.

26. The method of claim 20, wherein the organic solvent has a higher density than the aqueous solution.

27. The method of claim 20, wherein the hydrosomes have a higher refractive index from that of the organic solvent.

28. The method of claim 20 wherein droplets of the aqueous solution are generated by applying mechanical pressure by using an electromechanical transducer which generates a vibrational pressure wave within the aqueous solution to form a droplet of the aqueous solution by ejection from a droplet forming nozzles.

29. The method of claim 28, wherein the electomechanical transducer is fabricated from a piezoelectric material.

30. The method according to claim 25, wherein the perfluorinated compound is perfluoropolyether.

31. The method of claim 20, wherein the chemical component comprises DNA, proteins, enzymes, or fluorescent compounds.

32. The method of claim 20, wherein the chemical component is selected from the group consisting of genes, gene analogs, RNA, RNA analogs, DNA, DNA analogs, colloidal particles, receptors, receptor ligands, receptor antagonists, receptor blockers, enzymes, enzyme substrates, enzyme inhibitors, enzyme modulators, proteins, protein analogs, amino acids, amino acid analogs, peptides, peptide analogs, metabolites, metabolite analogs, oligonucleotides, oligonucleotide analogs, antigens, antigen analogs, haptens, hapten analogs, antibodies, antibody analogs, organelles, organelle analogs, cell nuclei, bacteria, viruses, gametes, inorganic ions, metal ions, metal clusters, polymers, fluorescent compounds and any combinations thereof.

33. The method of claim 23, wherein the focused beam of laser light includes light in the wavelength of about 300 nm to about 700 nm.

34. The method of claim 23, wherein the focused beam of laser light is continuous.

35. The method of claim 23, wherein the focused beam of laser light is pulsed.

36. The method of claim 23, wherein the focused beam of laser light is selected from the group consisting of the visible and ultraviolet wavelength ranges.

37. A system for creating microreactors, the system comprising:
a) a first container for holding an aqueous solution comprising at least one chemical component solubilized therein;
b) a second container for holding an organic solvent;
c) a droplet generator in fluid communication with the first and second container and positioned therebetween to generate droplets of the aqueous solution for ejection of same into the second container holding the organic solvent thereby generating hydrosomes; and
d) at least one source of electromagnetic energy positioned to direct electromagnetic energy at the second container to manipulate the hydrosomes for fusing together to form the microreactors, wherein the hydrosomes have a different refractive index from that of the organic solvent.

38. The system of claim 37, wherein the droplet generator comprises:
i) a plurality of nozzles in fluid communication with the first and second container wherein the nozzles comprise an aperture bore diameter sized to generate droplets of the aqueous solution; and
ii) a pressure producing means communicatively contacting the aqueous solution to cause an increased pressure within the aqueous solution thereby generating droplets of aqueous solution and causing the ejection of same through the nozzles into the second container holding the organic solvent thereby generating hydrosomes; and

39. The system of claim 37, wherein at least one source of electromagnetic energy comprises a focused beam of laser light in a wavelength range to form conditions for an optical trap.

40. The system of claim 39, wherein the focused beam of laser light includes light in the wavelength of about 300 nm to about 700 nm.

41. The system of claim 39, wherein the focused beam of laser light is continuous.

42. The system of claim 39, wherein the focused beam of laser light is pulsed.

43. The system of claim 39, wherein the focused beam of laser light is selected from the group consisting of the visible and ultraviolet wavelength ranges.

44. The system of claim 38, wherein the pressure producing means comprises a heating means to heat a portion of the aqueous solution to increase the pressure therein sufficiently to create a bubble in the aqueous solution thereby causing an expansion of the aqueous solution through the plurality of nozzles.

45. The system of claim 38, wherein the pressure producing means comprises a piezoelectric transducer that upon application of a voltage thereto, creates a vibration within the aqueous solution to displace the aqueous solution through the plurality of is nozzles thereby creating a droplet.

46. The system of claim 37, wherein the chemical component is selected from the group consisting of genes, gene analogs, RNA, RNA analogs, DNA, DNA analogs, colloidal particles, receptors, receptor ligands, receptor antagonists, receptor blockers, enzymes, enzyme substrates, enzyme inhibitors, enzyme modulators, proteins, protein analogs, amino acids, amino acid analogs, peptides, peptide analogs, metabolites, metabolite analogs, oligonucleotides, oligonucleotide analogs, antigens, antigen analogs, haptens, hapten analogs, antibodies, antibody analogs, organelles, organelle analogs, cell nuclei, bacteria, viruses, gametes, inorganic ions, metal ions, metal clusters, polymers, fluorescent compounds and any combinations thereof.

47. The system of claim 37, wherein the aqueous solution further comprises a surfactant.

48. The system of claim 47, wherein the surfactant is selected from the group consisting of t-octylphenoxypolyethoxyethanol, polyoxyethylenesorbitan monolaurate, polyoxyethylenesorbitan monopalmitate, polyoxyethylenesorbitan monostearate, polyoxyethylenesorbitan monooleate, polyoxyethylenesorbitan monotrioleate, (octylphenoxy)polyethoxyethanol, triethyleneglycol monolauryl ether and sorbitan monolaurate.

49. The system of claim 37, wherein the organic solvent is a perfluorinated compound.

50. The system of claim 37, wherein the organic solvent has a higher density than the aqueous solution.

51. The system of claim 37, wherein the hydrosomes have a higher refractive index from that of the organic solvent.

52. A system for creating hydrosomes and fusing same to create microreactors, the system comprising:
1) a first container for holding an aqueous solution comprising at least one chemical component solubilized therein and a surfactant;
2) a second container for holding an organic solvent;
3) a droplet generator in fluid communication with the first and second container and positioned therebetween, wherein the droplet generator device comprises:
i) a plurality of nozzles in fluid communication with the first and second container wherein the nozzles comprise an aperture bore diameter sized to generate droplets of the aqueous solution; and
ii) a pressure producing means communicatively contacting the aqueous solution to cause an increased pressure within the aqueous solution thereby generating droplets of aqueous solution and causing the ejection of same through the nozzles into the second container holding the organic solvent thereby generating hydrosomes; and
4) at least one source of electromagnetic energy positioned to direct electromagnetic energy at the second container to manipulate the hydrosomes for fusing together to form the microreactors.
